(19) United States
(12) Patent Application Publication
(10) Pub. No.: US 2004/0142961 A1
(43) Pub. Date: Jul. 22, 2004

(54) Utilization of substituted imidazo[1,2-a]-pyridine compounds in pharmaceutical formulations

(76) Inventors: Corinna Sundermann, Aachen (DE); Hagen-Heinrich Hennies, Simmerath (DE); Bernd Sundermann, Aachen (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/678,645

(22) Filed: Oct. 3, 2003

Related U.S. Application Data
(63) Continuation of application No. PCT/EP02/03795, filed on Apr. 5, 2002.

(30) Foreign Application Priority Data
Apr. 5, 2001 (DE) 101 17 183.8

(51) Int. Cl. A61K 31/4745

(52) U.S. Cl. 514/303

(57) ABSTRACT

Methods of using pharmaceutical formulations with imidazo[1,2-a]-pyridine compounds as active ingredients to inhibit nitric oxide synthase are disclosed. Treatments of certain conditions using imidazo[1,2-a]-pyridine compounds are also disclosed.

### CROSS-REFERENCE TO RELATED APPLICATIONS

This application is a continuation of International Patent Application No. PCT/EP02/03795, filed Apr. 5, 2002, designating the United States of America, and published in German as WO 02/080914, the entire disclosure of which is incorporated herein by reference. Priority is claimed based on Federal Republic of Germany Patent Application No. DE 101 17 183.8, filed Apr. 5, 2001.

### FIELD OF THE INVENTION

The present invention relates to the use of substituted imidazo[1,2-a]-pyridine compounds and their physiologically acceptable salts as inhibitors for nitric oxide synthase and for the preparation of pharmaceutical formulations.

### BACKGROUND OF THE INVENTION

Nitric oxide (NO) regulates numerous physiological processes, inter alia neurotransmission, relaxation and proliferation of the smooth musculature, adhesion and aggregation of thrombocytes and tissue injury and inflammation. Because of the large number of signal functions, a connection is made between nitric oxide and a number of diseases, for example in L. J. Ignarro, Angew. Chem. (1999), 111, pages 2002-2013 and in F. Murad, Angew. Chem. Int. Ed. (1999), 111, pages 1976-1989. The enzyme responsible for the physiological formation of nitric oxide, nitric oxide synthase (NO synthase), plays an important role here in therapeutic influencing of these diseases. Three different isoforms of NO synthase have so far been identified, that is to say the two constitutive forms nNO synthase and eNO synthase and the inducible form iNO synthase (A. J. Hobbs, A. Higgs, S. Moncada, Annu. Rev. Pharmacol. Toxicol. (1999), 39, pages 191-220; I. C. Green, P.-E. Chabrier, DDT (1999), 4, pages 47-49; P.-E. Chabrier et al., Cell. Mol. Life Sci. (1999), 55, pages 1029-1035).

The inhibition of NO synthase opens up new therapeutic procedures for various diseases associated with nitric oxide (A. J. Hobbs et al., Annu. Rev. Pharmacol. Toxicol. (1999), 39, pages 191-220; I. C. Green, P.-E. Chabrier, DDT (1999), 4, pages 47-49; P.-E. Chabrier et al., Cell. Mol. Life Sci. (1999), 55, pages 1029-1035), such as, for example, migraine (L. L. Thomsen, J. Olesen, Clinical Neuroscience (1998), 5, pages 28-33; L. H. Lassen et al., The Lancet (1997), 349, 401-402), septic shock, neurodegenerative diseases, such as multiple sclerosis, Parkinson's disease, Alzheimer's disease or Huntington's disease, inflammations, inflammatory pain, cerebral ischaemia, diabetes, meningitis and arteriosclerosis. The inhibition of NO synthase can moreover have an effect on wound healing, on tumors and on angiogenesis and cause a non-specific immunity to microorganisms (A. J. Hobbs et al., Annu. Rev. Pharmacol. Toxicol. (1999), 39, pages 191-220).

Active compounds known to date which inhibit NO synthase include, in addition to L-NMMA and L-NAME—i.e. analogues of L-arginine, from which nitric oxide and citrulline are formed in vivo with the participation of NO synthase—inter alia S-methyl-L-citrulline, aminoguanidine, S-methylisourea, 7-nitroindazole and 2-mercaptoethylguanidine (A. J. Hobbs et al., Annu. Rev. Pharmacol. Toxicol. (1999), 39, pages 191-220).

### SUMMARY OF THE INVENTION

An object of the present invention is to provide pharmaceutical formulations which act as an inhibitor on nitric oxide synthase.

Another object is to provide pharmaceutical formulations and methods suitable for treatment of migraine, septic shock, neurodegenerative diseases, such as multiple sclerosis, Parkinson's disease, Alzheimer's disease or Huntington's disease, inflammations, inflammatory pain, cerebral ischaemia, diabetes, meningitis, arteriosclerosis or for wound healing.

Other objects, advantages and novel features of the present invention will become apparent from the following detailed description of the invention when considered in conjunction with the accompanying drawings.

Surprisingly, it has now been found that substituted imidazo[1,2-a]-pyridine compounds corresponding to the following formula I act as inhibitors on nitric oxide synthase and are suitable in particular for treatment of migraine, septic shock, neurodegenerative diseases, such as multiple sclerosis, Parkinson's disease, Alzheimer's disease or Huntington's disease, inflammations, inflammatory pain, cerebral ischaemia, diabetes, meningitis, arteriosclerosis or for wound healing.

In one embodiment, the invention provides a method of inhibiting nitric oxide synthase in a mammal. The method comprises administering to the mammal an effective nitric oxide synthase inhibiting amount of at least one imidazo[1,2-a]-pyridine compound corresponding to formula I

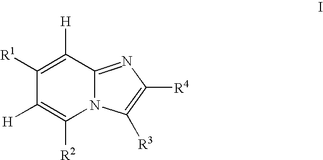

wherein,

R¹ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶, OH or OR⁷, preferably an unsubstituted or at least monosubstituted C₁₋₈-alkyl radical, F, Cl, Br, CN, NO₂, NH₂, C(═O)R⁵, CO₂H, CO₂R⁶, OH or OR⁷, particularly preferably an unsubstituted or at least monosubstituted C₁₋₈-alkyl radical,

R² represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶ or OH, preferably an unsubstituted or at least monosubstituted C₁₋₈-alkyl radical or H, particularly preferably H,

R³ represents an unsubstituted or at least monosubstituted C₁₋₈-alkyl radical, an unsubstituted or at least monosubstituted C₂₋₈-alkenyl, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, an unsubstituted or at least monosubstituted aryl or heteroaryl radical which is bonded via a C₁₋₈-alkylene group, CH₂SR⁸, CH₂OR⁸ or H, preferably an unsubstituted or at least monosubstituted C₁₋₈-alkyl radical or H, particularly preferably H,

R⁴ represents H, an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₇-heterocyclyl radical, an unsubstituted or at least monosubstituted aryl or heteroaryl radical, a C₃₋₈-cycloalkyl radical which is bonded via a C₁₋₈-alkylene group, a C₃₋₇-heterocyclyl radical which is bonded via a C₁₋₈-alkylene group, an unsubstituted or at least monosubstituted aryl or heteroaryl radical which is bonded via a C₁₋₈-alkylene group, preferably H, an unsubstituted or at least monosubstituted C₁₋₈-alkyl radical, an unsubstituted or at least monosubstituted aryl or heteroaryl radical or an unsubstituted or at least monosubstituted aryl or heteroaryl radical which is bonded via a C₁₋₈-alkylene group,

R⁵ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, a C₃₋₇-heterocyclyl radical, an unsubstituted or at least monosubstituted aryl or heteroaryl radical or an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical,

R⁶ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₄-alkylene group, an unsubstituted or at least monosubstituted aryl radical or an unsubstituted or at least monosubstituted aryl radical which is bonded via a C₁₋₈-alkylene group, preferably an unsubstituted or at least monosubstituted C₁₋₈-alkyl radical or an unsubstituted or at least monosubstituted aryl radical,

R⁷ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₄-alkylene group, an unsubstituted or at least monosubstituted aryl radical or an unsubstituted or at least monosubstituted aryl radical which is bonded via a C₁₋₈-alkylene group, preferably an unsubstituted or at least monosubstituted C₁₋₈-alkyl radical or an unsubstituted or at least monosubstituted aryl radical,

R⁸ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, an unsubstituted or at least monosubstituted aryl or heteroaryl radical, an unsubstituted or at least monosubstituted aryl or heteroaryl radical which is bonded via a C₁₋₈-alkylene group or a C₃₋₈-cycloalkyl radical, preferably an unsubstituted or at least monosubstituted C₁₋₈-alkyl radical or an unsubstituted or at least monosubstituted aryl or heteroaryl radical.

Preferably, said compound is in the form of its base or a physiologically acceptable salt.

Preferred C₁₋₈-alkyl radicals are selected from the group consisting of methyl, ethyl, n-propyl, 2-propyl, n-butyl, iso-butyl, sec-butyl, tert-butyl, n-pentyl, iso-pentyl, neo-pentyl, n-hexyl, 2-hexyl and n-octyl.

Preferred C₂₋₈-alkenyl radicals are selected from the group consisting of ethenyl (vinyl), propenyl (—CH₂CH═CH₂, —CH═CH—CH₃, —C(═CH₂)—CH₃), butenyl, pentenyl, hexenyl and octenyl.

Preferred C₂₋₈-alkinyl radicals are selected from the group consisting of ethinyl, propinyl (—CH—C≡CH, —C≡C—CH₃), butinyl, pentinyl, hexinyl and octinyl.

If the C₁₋₈-alkyl radical, the C₂₋₈-alkenyl radical or the C₂₋₈-alkinyl radical is present in a mono- or polysubstituted form, one or more hydrogen radical(s) is (are) preferably replaced by a substituent selected from the group consisting of F, Cl, Br, I, CN, NH₂, NH-alkyl, NH-aryl, NH-heteroaryl, NH-alkyl-aryl, NH-alkyl-heteroaryl, NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-aryl, S-heteroaryl, S-alkyl-aryl, S-alkyl-heteroaryl, S-heterocyclyl, S-alkyl-OH, S-alkyl-SH, OH, O-alkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl,

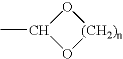

where n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NH-aryl, C(═O)NH-heterocyclyl, C(═O)N(alkyl)₂, C(═O)N(alkyl-aryl)₂, C(═O)N(alkyl-heteroaryl)₂, C(═O)N(heterocyclyl)₂, SO-alkyl, SO₂-alkyl, SO₂NH₂, SO₃H, cycloalkyl, aryl, heteroaryl and heterocyclyl, wherein polysubstituted C₁₋₈-alkyl radicals are to be understood as meaning those radicals which are poly-, e.g. di- or trisubstituted either on different atoms or on the same atom of the C₁₋₈-alkyl, C₂₋₈-alkenyl or C₂₋₈-alkinyl radical, for example trisubstituted on the same carbon atom, as in CF₃ or —CH₂CF₃, or on different atoms, as in —CH(OH)—CH═CH—CHCl₂. The polysubstitution can be by identical or by different substituents. If the substituent itself contains an alkyl group, this is preferably selected from the group consisting of methyl, ethyl, CH₂—OH and CF₃.

The expression “C₃₋₈-cycloalkyl radical” for the purposes of the present invention includes cyclic hydrocarbons having 3 to 8 carbon atoms, which can be saturated or unsaturated, unsubstituted or at least monosubstituted, wherein bonding of the cycloalkyl radical to the base skeleton of formula I can be via any desired ring member of the cycloalkyl radical. The C₃₋₈-cycloalkyl radical is preferably selected from the group consisting of cyclopropyl, cyclobutyl, cyclopentyl, cyclohexyl, cycloheptyl, cyclooctyl, cyclopentenyl, cyclohexenyl, cycloheptenyl and cyclooctenyl. The C₃₋₈-cycloalkyl radical is particularly preferably a cyclohexyl radical.

The expression “C₃₋₇-heterocyclyl radical” in the context of the present invention includes a 3-, 4-, 5-, 6- or 7-membered cyclic organic radical which contains at least 1, optionally also 2, 3, 4 or 5 heteroatoms in the ring system, wherein the heteroatoms can be identical or different and the cyclic radical is saturated or unsaturated but not aromatic and can be unsubstituted or at least monosubstituted. Bonding of the heterocyclyl radical to the base skeleton of formula I can be via any desired ring member of the heterocyclyl radical. The heterocyclyl radical can also be part of a bi- or polycyclic system. Preferred heteroatoms are selected from the group consisting of nitrogen, oxygen and sulfur. The C₃₋₇-heterocyclyl radical is preferably selected from the group consisting of tetrahydrofuryl, tetrahydropyranyl, pyrrolidinyl, piperidinyl, piperazinyl and morpholinyl.

The expression “aryl radical” in the context of the present invention denotes aromatic hydrocarbons, which can also be fused with further saturated, at least partly unsaturated or aromatic ring systems, wherein bonding of the aryl radical to the base skeleton of formula I can be via any desired ring member of the aryl radical. If the aryl radical contains more than one substituent, these can be identical or different and can be present in any desired and possible position of the aryl radical. The aryl radical is preferably selected from the group consisting of unsubstituted or at least monosubstituted phenyl, anthracenyl, 1-naphthyl and 2-naphthyl. The aryl radical is particularly preferably selected from the group consisting of phenyl, 3-hydroxyphenyl, 3-methoxyphenyl, 2,3-dihydroxyphenyl, 2,3-dimethoxyphenyl and 1-naphthyl.

The expression “heteroaryl radical” in the context of the present invention represents a 5-, 6- or 7-membered cyclic aromatic radical which contains at least 1, optionally also 2, 3, 4 or 5 heteroatoms, wherein the heteroatoms can be identical or different and wherein bonding to the base skeleton of formula I can be via any desired and possible ring member of the heteroaryl radical. If the heteroaryl radical contains more than one substituent, these heteroaryl substituents can be identical or different and can be present in any desired and possible position on the heteroaryl radical. The heterocyclic radical can also be fused with further saturated, at least partly unsaturated or aromatic ring systems. Preferred heteroatoms are selected from the group consisting of nitrogen, oxygen and sulfur. The heteroaryl radical is preferably selected from the group consisting of unsubstituted or at least monosubstituted pyrrolyl, furyl, thienyl, pyrazolyl, imidazolyl, pyridinyl, pyridazinyl, pyrimidinyl, pyrazinyl, pyranyl, indolyl, indazolyl, purinyl, pyrimidinyl, indolizinyl, quinolinyl, isoquinolinyl, quinazolinyl, carbazolyl, phenazinyl and phenothiazinyl. Particularly preferred heteroaryl radicals are selected from the group consisting of pyridin-2-yl, pyridin-3-yl, furan-2-yl, furan-3-yl, 5-hydroxymethylene-furan-2-yl, 5-nitro-furan-2-yl, 5-[1,3]-dioxolane-furan-2-yl, 5-carboxylic acid-furan-2-yl, thien-2-yl (2-thiophene), thien-3-yl (3-thiophene) and 5-carboxylic acid-2-thiophene (5-carboxylic acid-thien-2-yl).

If the C₃₋₈-cycloalkyl, the C₃₋₇-heterocyclyl, the aryl or the heteroaryl radical is mono- or polysubstituted, this is to be understood as meaning mono- or poly-, e.g. di-, tri- or tetrasubstitution of one or more hydrogen atoms of the ring system by a substituent selected from the group consisting of F, Cl, Br, I, CN, NH₂, NH-alkyl, NH-aryl, NH-heteroaryl, NH-alkyl-aryl, NH-alkyl-heteroaryl, NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-cycloalkyl, S-aryl, S-heteroaryl, S-alkyl-aryl, S-alkyl-heteroaryl, S-heterocyclyl, S-alkyl-OH, S-alkyl-SH, OH, O-alkyl, O-cycloalkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl

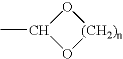

where n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NH-aryl, C(═O)NH-heterocyclyl, C(═O)N(alkyl)₂, C(═O)N(alkyl-aryl)₂, C(═O)N(alkyl-heteroaryl)₂, C(═O)N(heterocyclyl)₂, S(O)-alkyl, S(O)-aryl, SO₂-alkyl, SO₂-aryl, SO₂NH₂, SO₃H, CF₃, ═O, ═S; alkyl, cycloalkyl, aryl, heteroaryl and heterocyclyl, wherein a substituent can in turn be optionally substituted. The polysubstitution may be by identical or different substituents. For “aryl radicals”, particularly preferred substituents are selected from the group consisting of F, CF₃, OH and O—CH₃. For “heteroaryl radicals”, particularly preferred substituents are selected from the group consisting of OH, O—CH₃, CH₂OH, NO₂, CO₂H, CO₂ethyl and [1,3]-dioxolane. For “cycloalkyl radicals”, particularly preferred substituents include CO₂H or CO₂ethyl.

The use of at least one compound selected from the group consisting of

2-(4-methoxy-phenyl)-7-methyl-imidazo[1,2-a]pyridine,

2,7-dimethyl-imidazo[1,2-a]pyridine,

7-methyl-imidazo[1,2-a]pyridine and

2-tert-butyl-7-methyl-imidazo[1,2-a]pyridine,

in the form of a base or a physiologically acceptable salt, preferably in the form of the hydrochloride, as an inhibitor of nitric oxide synthase is very particularly preferred.

If the substituted imidazo[1,2-a]-pyridine compounds of formula I employed according to the invention or physiologically acceptable salts thereof contain at least one asymmetric center, they can exist in the form of their racemates, their pure enantiomers, their pure diastereomers or in the form of a mixture of at least two of the abovementioned stereoisomers. The substituted imidazo[1,2-a]-pyridine compounds of formula I can also exist in the form of a mixture of their enantiomers or diastereomers. These mixtures may contain two or more of the particular stereoisomers in any desired mixing ratio. Chiral substituted imidazo[1,2-a]-pyridine compounds of formula I in enantiomerically pure form are preferably used.

The substituted imidazo[1,2-a]-pyridine compounds of formula I can be prepared by conventional methods known to persons skilled in the art.

The preparation of the compounds of formula I employed according to the invention is preferably carried out by reaction of a substituted 2-aminopyridine corresponding to formula II, wherein R¹ and R² have the meanings given above for formula I,

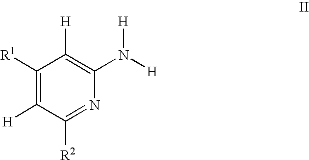

preferably in solution, with an α-halogenocarbonyl compound corresponding to formula III

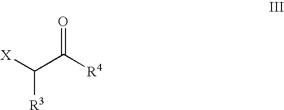

wherein the radicals R³ and R⁴ have the meanings given above formula I, and X represents halogen, preferably Cl, Br or I, water and hydrogen halide being split off.

The process for the preparation of the compounds of formula I according to the invention is advantageously carried out under conditions under which water and/or hydrogen halide are preferably removed continuously from the reaction mixture.

Hydrogen halide can preferably be scavenged by addition of soluble or insoluble organic or inorganic bases and removed from the reaction mixture in this way.

Water can preferably be removed from the reaction mixture by azeotropic distillation or by addition of drying agents or hygroscopic substances.

The preparation of the compounds of formula I according to the invention, by the above process, at temperatures of more than 100° C., with or without a solvent, represents a further possibility for removing water from the reaction mixture.

The preparation of the compounds of formula I according to the invention, by reaction of substituted 2-aminopyridines of formula II with α-halogenocarbonyl compounds of formula III, wherein X represents Br, in boiling anhydrous ethanol is particularly preferred.

The preparation of the compounds of formula I according to the invention, by reaction of substituted 2-aminopyridines of formula II with α-halogenocarbonyl compounds of formula III, wherein X represents Br or Cl, in boiling anhydrous methylene chloride or chloroform using a water separator is also preferred.

The substituted 2-aminopyridines of formula II and the α-halogenocarbonyl compounds of formula III are generally commercially available or can be prepared by conventional methods known to persons skilled in the art.

The substituted imidazo[1,2-a]-pyridine compounds of formula I employed according to the invention can be isolated either as a free base or as a salt after the process employed for their preparation. The free base of the particular compound of formula I is usually obtained after the reaction has been carried out following the process according to the invention described above and optionally subsequent working up by conventional methods known to persons skilled in the art. The free base, obtained in this way or formed in situ without isolation, of the particular compound of formula I can then be converted into the corresponding physiologically acceptable salt, for example by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid.

Conversion of a particular compound of formula I into the corresponding hydrochloride can preferably also be achieved by adding trimethylsilyl chloride (TMSCl) to the compound of formula I, as the free base, dissolved in a suitable organic solvent, such as, e.g. butan-2-one (methyl ethyl ketone).

If the substituted imidazo[1,2-a]-pyridine compound of formula I according to the invention is obtained in the form of a racemate or other mixture of its various enantiomers and/or diastereomers by the preparation process according to the invention, these can be separated and optionally isolated by conventional processes known to persons skilled in the art. Examples of such processes include chromatographic separation processes, in particular liquid chromatography processes under normal pressure or under elevated pressure, preferably MPLC and HPLC processes, and processes of fractional crystallization. In this procedure, in particular, individual enantiomers, e.g. diastereomeric salts can be separated from one another by HPLC on a chiral phase or by crystallization with chiral acids, for example (+)-tartaric acid, (−)-tartaric acid or (+)-10-camphorsulfonic acid.

The present invention also relates to the use of at least one substituted imidazo[1,2-a]-pyridine compound of formula I as an inhibitor of nitric oxide synthase and/or in pharmaceutical formulations for treatment of migraine, septic shock, neurodegenerative diseases, preferably multiple sclerosis, Parkinson's disease, Alzheimer's disease or Huntington's disease, inflammatory pain, cerebral ischaemia, diabetes, meningitis, arteriosclerosis or for wound treatment.

The present invention also relates to the use of at least one substituted imidazo[1,2-a]-pyridine compound of formula I, with the proviso that the radicals R³ and R⁴ do not both represent a 4-methoxy-phenyl radical if the radicals R¹ and R², which are identical or different, represent a C₁₋₄-alkyl radical, a C₁₋₄-alkoxy radical, an OH radical or an NO₂ radical, as an inhibitor of nitric oxide synthase and/or in pharmaceutical formulations for treatment of inflammations.

The present invention also relates to the use of at least one substituted imidazo[1,2-a]-pyridine compound of formula I for the preparation of a pharmaceutical formulation for treatment of migraine, septic shock, neurodegenerative diseases, preferably multiple sclerosis, Parkinson's disease, Alzheimer's disease or Huntington's disease, inflammatory pain, cerebral ischaemia, diabetes, meningitis, arteriosclerosis or for wound treatment.

The present invention also relates to the use of at least one substituted imidazo[1,2-a]-pyridine compound of formula I, with the proviso that the radicals R³ and R⁴ do not both represent a 4-methoxy-phenyl radical if the radicals R¹ and R², which are identical or different, represent a C₁₋₄-alkyl radical, a C₁₋₄-alkoxy radical, an OH radical or an NO₂ radical, for the preparation of a pharmaceutical formulation for treatment of inflammations.

The pharmaceutical formulations used in the invention can exist as liquid, semi-solid or solid pharmaceutical formulation forms, for example in the form of injection solutions, drops, juices, syrups, sprays, suspensions, granules, tablets, patches, capsules, plasters, suppositories, ointments, creams, lotions, gels, emulsions, aerosols or in multiparticulate form, for example in the form of pellets or granules, and can also be administered as such.

In addition to at least one substituted imidazo[1,2-a]-pyridine compound of formula I, the pharmaceutical formulations according to the invention typically comprise further conventional physiologically acceptable pharmaceutical auxiliary substances known to persons skilled in the art, which are preferably selected from the group consisting of carriers, fillers, solvents, diluents, surface-active agents, dyestuffs, preservatives, disintegrating agents, lubricants, greasing agents, flavorings and binders.

The choice of the physiologically acceptable auxiliary substances and the amounts thereof to be employed depend on whether the pharmaceutical formulation is to be administered orally, subcutaneously, parenterally, intravenously, intraperitoneally, intradermally, intramuscularly, intranasally, buccally, rectally or locally, for example on infections on the skin, the mucous membranes and on the eyes. Formulations in the form of tablets, coated tablets, capsules, granules, pellets, drops, juices and syrups are preferable for oral administration, and solutions, suspensions, easily reconstitutable dry formulations and sprays are suitable for parenteral, topical and inhalatory administration. Compounds of formula I in a depot in dissolved form or in a plaster, optionally with the addition of agents which promote penetration through the skin, are suitable formulations for percutaneous administration. Formulation forms which can be used orally or percutaneously can also release the compounds of formula I in a delayed manner.

The pharmaceutical formulations are prepared with the aid of conventional means, devices, methods and processes known to persons skilled in the art, such as are described, for example, in “Remington's Pharmaceutical Sciences”, ed. A. R. Gennaro, 17th ed., Mack Publishing Company, Easton, Pa. (1985), in particular in part 8, chapter 76 to 93. The corresponding literature description is incorporated herein by reference and thus forms part of the disclosure.

The amount of the particular compound of formula I to be administered to the patient can vary and depends, for example, on the weight or the age of the patient and on the mode of administration, on the indication and on the severity of the disease. 0.1 to 5,000 mg/kg, preferably 1 to 500 mg/kg, particularly preferably 2 to 250 mg of at least one compound of formula I are conventionally administered per kg of body weight of the patient.

Molecular Pharmacology Studies

The assays used to determine the inhibition of nitric oxide synthase by the compounds of formula I employed according to the invention are described in the following text:

Nitric Oxide Synthase Assay

This assay allows the determination of the percentage inhibition of NO synthase by a compound of formula I employed according to the invention by measuring the NO synthase activity under the action of the compound. In this procedure, NO synthase is mixed together with radioactively labelled arginine and the particular compound of formula I under suitable conditions. After interruption of the NO formation reaction at a given point in time, the amount of unreacted arginine is determined directly or indirectly. Comparison of this amount with the amount of arginine remaining from the mixture of NO synthase and arginine free of the test compound of formula I and under otherwise identical conditions gives the percentage inhibition of NO synthase by the test compound. This assay can be carried out as follows:

(a) incubation of NO synthase with labelled arginine as the substrate in a reaction vessel,

(b) separation of the labelled arginine from labelled citrulline formed as the product of the enzymatic reaction at a point in time at which the concentration of citrulline is increasing, and

(c) measurement of the amount of arginine separated in each case.

The separation is carried out through a filter plate membrane.

This NO synthase assay is particularly suitable for a “high throughput screening” (HTS) on microtiter plates (MTP).

HTS NO Synthase Assay: General Procedure

In this HTS NO synthase assay, radioactive arginine is used as the substrate. The assay volume can be chosen in the range between 25 μl and 250 μl, depending on the nature of the microtiter plate (MTP). Cofactors and coenzymes are added, depending on the enzyme source used. The incubation of the batches in this microtiter plate (assay MTP) according to step (a) is carried out at room temperature for between 5 and 60 minutes, depending on the enzyme activity (units) used. At the end of the incubation (step (a)), the plate is placed in a cell harvester equipped with an MTP which has a cation exchanger membrane as the filter base (filter MTP). All the batches of the assay MTP are transferred into this filter MTP and filtered with suction over a cation exchanger filter plate, i.e., a filter paper loaded with phosphate groups. The filter MTP is then washed with buffer or water. Using this procedure, the arginine substrate which remains is bound to the cation exchanger, while the radioactive citrulline formed enzymatically is washed out quantitatively. After drying of the filter MTP and addition of scintillation liquid, the bound arginine can be counted on a scintillation counter. An NO synthase reaction which has not been inhibited is reflected in a low radioactivity. An inhibited enzyme reaction means that the radioactive arginine has not been reacted. That is to say, a high radioactivity is found on the filter.

Materials Used

Arginine, L-[2,3,4-³H]-monohydrochloride; order no. NET-1123, NEN

CaCl₂ anhydrous; order no. 2388.1000; Merck KGaA

1,4-Dithiothreitol (DTT), order no. 708984; ROCHE

Na₂EDTA dihydrate; order no. 03680; FLUKA

HEPES, order no. H-3375; SIGMA

NADPH, tetrasodium salt; order no. 1585363; ROCHE

TRIS; ORDER No. 93349; FLUKA

| | |
|---|---|
| Enzyme preparation buffer: | 50 mM Tris-HCl with 1 mM EDTA: The |
| | pH of the buffer was adjusted to 7.4 at |
| | 4° C. |
| Incubation buffer | 50 mM HEPES with 1 mM EDTA; 1.25 mM |
| (medium): | CaCl₂ and 1 mM dithiothreitol. |
| | The pH of the buffer was adjusted to 7.4 |
| | at 25° C. |
| Washing medium: | H₂O |

For purposes of clarity, EDTA in the materials list above means ethylenediamine tetra-acetic acid. HEPES means 4-(2-hydroxyethyl)-1-piperazineethanesulfonic acid. NADPH means nicotinamide adenine dinucleotide phosphate. Tris means tris(hydroxymethyl)aminomethane.

Enzyme Preparation

Rat cerebelli were used as the starting tissue. The animals were narcotized and sacrificed, the brain tissue, the cerebellum, was removed, 1 ml enzyme preparation buffer (4° C.) was added per rat cerebellum, and the tissue was broken down with a Polytron homogenizer for 1 min at 6,000 rpm. Thereafter, centrifugation was carried out at 4° C. for 15 min at 20,000 g, and the supernatant was then decanted and frozen in portions at −80° C. (precipitate discarded).

Incubation Batch:

96-well MTP with a “well” capacity of≦250 μl were used Pipetting sequence: see table 1:

TABLE 1

| Substance | Molarity i.b. | μl | *Protein i.b. |
|---|---|---|---|
| Incubat. buffer | — | 100 | — |
| Test substance | variable; | variable; | — |
| | preferably 10⁻⁵ M | preferably 20 μl | |
| NADPH | 0.5 mM | 20 | — |
| Enzyme | — | variable; | variable; |
| (see example 3) | | maximum | maximum |
| | | volume of the | amount of protein |
| | | enzyme | which can be |
| | | solution = 50 μl | employed = |
| | | | 100 μg |
| [³H]substrate | variable; | variable; | — |
| | preferably 50 nM | preferably 10 μl | |

| | |
|---|---|
| End volume: | max. 250 μl |

*The protein determination was carried out by the method of O. H. Lowry et al; J. Biol. Chem. 193, 265 (1951). The corresponding literature description is incorporated herein by reference and forms part of the disclosure.
i.b. = in the batch

When the pipetting operation had ended, a cover was placed over this MTP (assay MTP). Incubation at 25° C. (room temperature (RT)) for 5-60 min, depending on the amount and activity of the enzyme employed.

The content of the assay MTP was then transferred with the aid of a 96-well cell harvester into a 96-well cation exchanger MTP (filter MTP) and filtered with suction. A single washing with 200 ml H₂O (from a trough) followed.

The plate was then dried for 1 hour at 60° C. in a drying cabinet. The bottom of the filter MTP was then sealed with a “back seal” from underneath. Thereafter 35 μl of scintillator were pipetted in per well. The upper side of the plate was furthermore sealed with a “top seal”. After a waiting time of 1 hour, the plate was measured on a β-counter.

In HTS operation, the incubation medium, NADPH solution and enzyme solution were combined before the start of the pipetting step, so that three separate pipettings did not have to be carried out in a time-consuming manner.

Citrulline Assay

This assay was carried out as described by D. S. Bredt and S. H. Snyder (Proc. Natl. Acad. Sci. USA (1990), 87, 682-685). The corresponding literature description is incorporated herein by reference and forms part of the present disclosure.

The invention is explained in the following text with the aid of examples. These explanations are provided merely as examples and are not intended to, nor should they be understood to, be limiting.

### EXAMPLES

### Example 1

### 2-(4-Methoxy-phenyl)-7-methyl-imidazo[1,2-a]pyridine

1.50 g 2-amino-4-methylpyridine were dissolved in 30 ml analytical grade ethanol; 3.18 g 2-bromo-4′-methoxyacetophenone were added, and the reaction mixture was heated under reflux for two hours and subsequently stirred overnight at a temperature of 20 to 25° C. For working up, the reaction mixture was concentrated to dryness in vacuo, the residue was taken up in methylene chloride and two-molar aqueous hydrochloric acid, and the phases were separated. Five per cent sodium hydroxide solution was added to the very cloudy organic phase until two clear phases were obtained. The clear phases were separated, the aqueous phase was extracted again with methylene chloride, and the organic phases were combined, dried over sodium sulfate and concentrated. The resulting crude product (2.90 g) was dissolved in 23 ml 2-butanone, and the hydrochloride was precipitated by addition of 120 μl water followed by 1.69 ml chlorotrimethylsilane and subsequent stirring overnight. The yield of 2-(4-methoxy-phenyl)-7-methyl-imidazo[1,2-a]pyridine hydrochloride was 2.63 g (corresponding to 69% of the theoretical amount).

### Example 2

### 2,7-Dimethyl-imidazo[1,2-a]pyridine

1.50 g 2-amino-4-methylpyridine were dissolved in 50 ml analytical grade ethanol, 2.57 g 1-chloropropan-2-one were added and the reaction mixture was heated under reflux for two hours and subsequently stirred overnight at a temperature of 20 to 25° C. For working up, the reaction mixture was concentrated to dryness in vacuo, the residue was taken up in methylene chloride and two-molar aqueous hydrochloric acid, and the phases were separated. The aqueous phase was rendered basic with five per cent sodium hydroxide solution and extracted twice with ether, and the ether extracts were combined, dried over sodium sulfate and concentrated. The resulting crude product (1.44 g) was dissolved in 12 ml 2-butanone, and the hydrochloride was precipitated by addition of 97 μl water followed by 1.37 ml chlorotrimethylsilane and subsequent stirring overnight. The yield of 2,7-dimethyl-imidazo[1,2-a]pyridine hydrochloride was 1.68 g (corresponding to 66% of the theoretical amount).

### Example 3

### 7-Methyl-imidazo[1,2-a]pyridine

1.50 g 2-amino-4-methylpyridine were dissolved in 50 ml methylene chloride; 4.84 g of a 45% by weight aqueous chloroacetaldehyde solution were added, and the reaction mixture was heated under reflux overnight using a water separator. For working up, 2N hydrochloric acid and methylene chloride were added to the reaction mixture; the phases were separated; the aqueous phase was rendered basic with five per cent sodium hydroxide solution and extracted twice with ether, and the ether extracts were combined, dried over sodium sulfate and concentrated. The crude product (1.42 g) obtained was dissolved in 12 ml 2-butanone and the hydrochloride was precipitated by addition of 106 μl water followed by 1.50 ml chlorotrimethylsilane and subsequent stirring overnight. The yield of 7-methyl-imidazo[1,2-a]pyridine hydrochloride was 1.59 g (corresponding to 67% of the theoretical amount).

### Example 4

### 2-tert-Butyl-7-methyl-imidazo[1,2-a]pyridine

1.50 g 2-amino-4-methylpyridine were dissolved in 30 ml analytical grade ethanol; 2.48 g 1-bromo-3,3-dimethyl-butan-2-one were added, and the reaction mixture was heated under reflux for two hours and subsequently stirred overnight at a temperature of 20 to 25° C. For working up, the reaction mixture was concentrated to dryness in vacuo; the residue was taken up in methylene chloride and two-molar aqueous hydrochloric acid, and the phases were separated. The aqueous phase was rendered basic with five per cent sodium hydroxide solution and extracted twice with ether, and the ether extracts were combined, dried over sodium sulfate and concentrated. The resulting crude product (1.84 g) was dissolved in 14 ml 2-butanone, and the hydrochloride was precipitated by addition of 89 μl water followed by 1.26 ml chlorotrimethylsilane and subsequent stirring overnight. The yield of 2-tert-butyl-7-methyl-imidazo[1,2-a]pyridine hydrochloride was 2.12 g (corresponding to 69% of the theoretical amount).

Molecular Pharmacology Study

The compounds prepared according to examples 1 to 4 were tested in the HTS NO synthase assay as described above. The inhibition of nitric oxide synthase (10 μM) by the compounds according to the examples is shown in the following Table 2:

TABLE 2

| | Inhibition of nitric oxide synthase (10 μM) |
|---|---|
| Example no.: | in per cent |
| 1 | 39 |
| 2 | 68 |
| 3 | 53 |
| 4 | 89 |

The foregoing description and examples have been set forth merely to illustrate invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of inhibiting nitric oxide synthase in a mammal, said method comprising administering to said mammal an effective nitric oxide synthase inhibiting amount of at least one imidazo[1,2-a]-pyridine compound corresponding to formula I 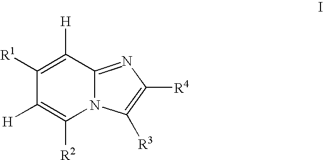
wherein,
R¹ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶, OH or OR₇;
R² represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶ or OH;
R³ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, an unsubstituted or at least monosubstituted aryl or heteroaryl radical which is bonded via a C₁₋₈-alkylene group, CH₂SR⁸, CH₂OR⁸ or H;
R⁴ represents H, an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, an unsubstituted or at least monosubstituted aryl or heteroaryl radical, or an unsubstituted or at least monosubstituted aryl or heteroaryl radical which is bonded via a C₁₋₈-alkylene group;
R⁵ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, a C₃₋₇-heterocyclyl radical, an unsubstituted or at least monosubstituted aryl or heteroaryl radical or an unsubstituted or at least monosubstituted aryl or heteroaryl radical which is bonded via a C₁₋₈-alkylene group;
R⁶ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical or an unsubstituted or at least monosubstituted aryl or heteroaryl radical which is bonded via a C₁₋₈-alkylene group;
R⁷ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical or an unsubstituted or at least monosubstituted aryl or heteroaryl radical which is bonded via a C₁₋₈-alkylene group; and
R⁸ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, an unsubstituted or at least monosubstituted aryl or heteroaryl radical, an unsubstituted or at least monosubstituted aryl or heteroaryl radical which is bonded via a C₁₋₈-alkylene group or a C₃₋₈-cycloalkyl radical,
or a salt thereof with a physiologically acceptable acid.

2. A method according to claim 1, wherein said compound is present in the form of a free base.

3. A method according to claim 1, wherein R¹ represents an unsubstituted or at least monosubstituted C₁₋₈-alkyl radical, F, Cl, Br, CN, NO₂, NH₂, C(═O)R⁵, CO₂H, CO₂R⁶, OH or OR⁷.

4. A method according to claim 1, wherein R¹ represents an unsubstituted or at least monosubstituted C₁₋₈-alkyl radical.

5. A method according to claim 1, wherein R² represents H.

6. A method according to claim 1, wherein R² represents an unsubstituted or at least monosubstituted C₁₋₈-alkyl radical.

7. A method according to claim 1, wherein R³ represents H.

8. A method according to claim 1, wherein R³ represents an unsubstituted or at least monosubstituted C₁₋₈-alkyl radical.

9. A method according to claim 1, wherein R⁴ represents H, an unsubstituted or at least monosubstituted C₁₋₈-alkyl radical, an unsubstituted or at least monosubstituted aryl or heteroaryl radical or an unsubstituted or at least monosubstituted aryl or heteroaryl radical which is bonded via a C₁₋₈-alkylene group.

10. A method according to claim 1, wherein R⁵ represents an unsubstituted or at least monosubstituted C₁₋₈-alkyl radical or an unsubstituted or at least monosubstituted aryl or heteroaryl radical.

11. A method according to claim 1, wherein R⁶ represents an unsubstituted or at least monosubstituted C₁₋₈-alkyl radical or an unsubstituted or at least monosubstituted aryl radical.

12. A method according to claim 1, wherein R⁷ represents an unsubstituted or at least monosubstituted C₁₋₈-alkyl radical or an unsubstituted or at least monosubstituted aryl radical.

13. A method according to claim 1, wherein R⁸ represents an unsubstituted or at least monosubstituted C₁₋₈-alkyl radical or an unsubstituted or at least monosubstituted aryl or heteroaryl radical.

14. A method according to claim 1, wherein said at least one imidazo[1,2-a]-pyridine compound is selected from the group consisting of
2-(4-methoxy-phenyl)-7-methyl-imidazo[1,2-a]pyridine,
2,7-dimethyl-imidazo[1,2-a]pyridine,
7-methyl-imidazo[1,2-a]pyridine,
2-tert-butyl-7-methyl-imidazo[1,2-a]pyridine, and
salts of any of the foregoing with a physiologically acceptable acid.

15. A method according to claim 14, wherein said at least one imidazo[1,2-a]-pyridine compound is present in the form of a free base.

16. A method of treating a condition selected from the group consisting of migraine, septic shock, multiple sclerosis, Alzheimer's disease, inflammatory pain, diabetes, meningitis, or a wound in a mammal, said method comprising administering to said mammal an effective amount of a compound according to claim 1.

17. A method according to claim 16, wherein said condition is migraine.

18. A method according to claim 16, wherein said condition is septic shock.

19. A method according to claim 16, wherein said condition is multiple sclerosis.

20. A method according to claim 16, wherein said condition is Alzheimer's disease.

21. A method according to claim 16, wherein said condition is inflammatory pain.

22. A method according to claim 16, wherein said condition is diabetes.

23. A method according to claim 16, wherein said condition is meningitis.

24. A method according to claim 16, wherein said condition is a wound.
